(19) United States
(12) Patent Application Publication
(10) Pub. No.: US 2010/0137838 A1
(43) Pub. Date: Jun. 3, 2010

(54) NON-PVC SYSTEM TUBE FOR BIOMEDICAL

(75) Inventor: Young-Kuk Hwang, Hwaseong-si (KR)

Correspondence Address: HOLME ROBERTS & OWEN LLP, 1700 LINCOLN STREET, SUITE 4100, DENVER, CO, 80203, US

(73) Assignee: CHOONGWAE CORPORATION, Choongnam-Do (KR)

(21) Appl. No.: 12/595,070

(22) PCT Filed: Apr. 11, 2008
(86) PCT No.: PCT/KR2008/002072

§ 371 Date: Oct. 8, 2009

(30) Foreign Application Priority Data
Apr. 11, 2007 (KR) 10-2007-0035337

(51) Int. Cl. A61M 25/00; A61M 39/00

(52) U.S. Cl. 604/533; 604/408

(57) ABSTRACT

The present invention relates to a medical tube, comprising an inner layer comprising 50 to 90% by weight of polypropylene-based elastomer and 10 to 50% by weight of polypropylene, based on the total weight of the inner layer; an intermediate layer comprising 45 to 55% by weight of polypropylene-based elastomer and 45 to 55% by weight of polypropylene, based on the total weight of the intermediate layer; and an outer layer comprising 20 to 55% by weight of polypropylene-based elastomer and 45 to 80% by weight of polypropylene, based on the total weight of the outer layer.

### TECHNICAL FIELD

The present invention relates to a NON-PVC tube for medical use.

### BACKGROUND ART

A medical tube includes an object type connected to a medical fluid bag and any foreign object type that may be inserted into a patient's body for any purpose, including medical, diagnostic and/or therapeutic purposes. Further, the medical tube includes a port used for connecting the tube to the medical fluid bag or separating the tube from the medical fluid bag.

In general, the medical fluid bag refers to containers capable of containing any fluid which is used for medical purposes, such as an IV (intravenous) solution and blood. The medical fluid bag has a tube that one end portion of the tube is connected to the bag for the influx of fluid therein and the other end portion of the tube is sealed with seal materials. As the seal material, a plastic is mainly used, and a medical tip may also be connected to the other end portion of the tube for seal of the tube. The medical tip may include a rubber stopper or not.

For medical purposes, the tube is required to have several properties, in particular, good adhesiveness to the material of medical fluid bags. Since use of NON-PVC materials as the material for medical fluid bags is recently required, there is a need to develop a medical tube capable of easily bonding to the NON-PVC materials, while having no less physical properties such as transparency, heat resistance, and flexibility than PVC tube.

### DISCLOSURE

### Technical Problem

It is an object of the present invention to provide a medical tube which has more excellent transparency, flexibility, heat resistance, and chemical stability than PVC tubes, and has excellent heat adhesiveness to NON-PVC medical fluid bags and gas-barrier property.

### Technical Solution

The present invention provides a medical tube comprising an inner layer comprising 50 to 90% by weight of polypropylene-based elastomer and 10 to 50% by weight of polypropylene, based on the total weight of the inner layer;

an intermediate layer comprising 45 to 55% by weight of polypropylene-based elastomer and 45 to 58% by weight of polypropylene, based on the total weight of the intermediate layer; and

an outer layer comprising 20 to 55% by weight of polypropylene-based elastomer and 45 to 80% by weight of polypropylene, based on the total weight of the outer layer.

### ADVANTAGEOUS EFFECTS

The medical tube of the present invention is excellent in transparency, flexibility, heat resistance, and chemical stability, and also has excellent heat adhesiveness to NON-PVC medical fluid bags and gas-barrier property.

### DESCRIPTION OF DRAWINGS

FIG. 1 is a schematic cross-sectional view of the medical tube according to one embodiment of the present invention; and

FIG. 2 is a schematic view of a medical fluid bag which is generally used.

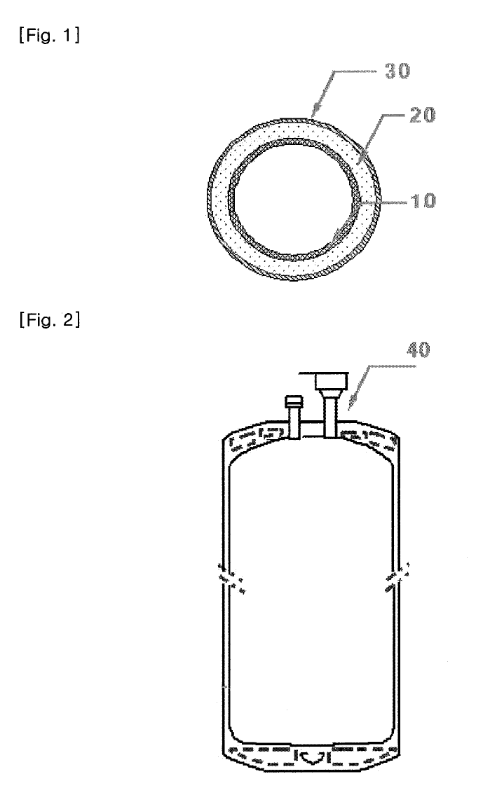

### BEST MODE

The present invention relates to a medical tube comprising

an inner layer comprising 50 to 90% by weight of polypropylene-based elastomer and 10 to 50% by weight of polypropylene, based on the total weight of the inner layer;

an intermediate layer comprising 45 to 55% by weight of polypropylene-based elastomer and 45 to 58% by weight of polypropylene, based on the total weight of the intermediate layer; and

an outer layer comprising 20 to 55% by weight of polypropylene-based elastomer and 45 to 80% by weight of polypropylene, based on the total weight of the outer layer.

With respect to the medical tube of the present invention, the inner layer may further comprise 1 to 40% by weight of polyethylene. In addition, the outer layer may further comprise 1 to 20% by weight of polyethylene.

With respect to the medical tube of the present invention, it is preferable that the inner layer, intermediate layer, and outer layer have a different composition ratio from each other, for the purpose of satisfying physical properties which are required for each layer.

The medical tube of the present invention is excellent in transparency, flexibility, heat resistance, and chemical stability, and also has excellent heat adhesiveness to NON-PVC medical fluid bags and gas-barrier property, thereby being very useful in medical field.

As used herein, the term “polypropylene-based elastomer” refers to a resin having polypropylene and a thermoplastic elastomer as main ingredients. The thermoplastic elastomer may be exemplified by styrene block copolymers (SBS, SIS, hydrogen-added SBC), olefin-based elastomers (non-crosslinked/crosslinked), SBC compounds (SBS, hydrogen-added SBC, non-crosslinked/crosslinked), vinyl chloride-based elastomers (TPVC), chlorinated polyethylene elastomers (CPE), urethane-based elastomers (TPU), polyester-based elastomers (TPEE), polyamide-based elastomer (TPAE), fluorine-based elastomers, chlorinated ethylene copolymer crosslinked alloy, silicone-based elastomers, syndiotactic 1,2-polybutadiene, and ester halogen-based polymer alloy.

With respect to the medical tube of the present invention, the content of ingredients constituting the inner layer is preferably 50 to 90% by weight of polypropylene-based elastomer and 10 to 50% by weight of polypropylene. Since the inner layer is a portion where a medical tip is inserted thereto, the composition ratio indicates a preferred range for the connection of the tip and the prevention of the tip separation upon using the product. That is, in the case where the content of polypropylene-based elastomer is less than 50% by weight, the flexibility becomes low to reduce the possibility of trouble with equipment for production of a medical tube, but there are problems in the insertion of the tip and in the prevention of the tip separation. In the case where the content of polypropylene-based elastomer is more than 90% by weight, the flexibility is greatly increased to generate troubles with the equipment for the production of a medical tube.

In the case where the content of polypropylene is less than 10% by weight, the content of polypropylene-based elastomer is relatively increased to excessively increase flexibility, thereby causing troubles with the equipment for the production of a medical tube and separation of the medical tube from the medical fluid bag. In the case where the content of polypropylene is more than 50% by weight, the content of polypropylene-based elastomer is relatively decreased to generate functional problems in the insertion of the medical tip and in the prevention of the tip separation.

With respect to the medical tube of the present invention, the inner layer preferably consists of 50 to 80% by weight of polypropylene-based elastomer and 20 to 50% by weight of polypropylene; or more preferably 1 to 40% by weight of polyethylene, in addition to 50 to 70% by weight of polypropylene-based elastomer and 10 to 30% by weight of polypropylene, based on the total weight of the inner layer. The composition ratio has the same meaning as mentioned above. The composition ratio is provided only for the purpose of illustrating more preferred range of composition ratio.

In particular, upon using the medical tip, if the content of polyethylene is within the above range, the medical tube exhibits more excellent physical properties, in terms of the insertion of the medical tip and the prevention of the tip separation, as compared to the case of using polypropylene only. Accordingly, in the case where the content of polyethylene is less than 1% by weight, its effect due to the addition is not sufficient. In the case where the content of polyethylene is more than 40% by weight, the effect is no longer increased, whereas heat resistance required for sterilization is deteriorated, and the problems related to relative decrease of the polypropylene content are generated.

With respect to the medical tube of the present invention, the intermediate layer functions to increase the flexibility of the medical tube and to connect the inner layer and the outer layer by adhesion. The content of ingredient consisting of the intermediate layer is preferably 45 to 55% by weight of polypropylene-based elastomer and 45 to 58% by weight of polypropylene. In the case where the content of the polypropylene-based elastomer is less than 45% by weight, the content of polypropylene is relatively high to reduce the flexibility of the tube. In the case where the content of the polypropylene-based elastomer is more than 55% by weight, the flexibility of the intermediate layer is greatly increased to generate an adverse effect on hardness of the tube. Further, in the case where the content of the polypropylene is less than 45% by weight, hardness of the intermediate layer becomes too weak to ensure the hardness of the tube. In the case where the content of the polypropylene is more than 58% by weight, the content of polypropylene-based elastomer is relatively low to reduce the flexibility of the tube.

With respect to the medical tube of the present invention, the outer layer functions to control the processability of the tube, which is associated with workability in the equipment for the production of the tube. For example, in the case where the content of polypropylene (PP) in the outer layer is low, the flexibility is too high to perform continuous operation in a feeding apparatus. In addition, the tube of the present invention is used connecting with the medical fluid bags, and the medical fluid bags are generally prepared using a polyolefin-based resin plastic film. Accordingly, the outer layer of the medical tube of the present invention is prepared by using a high content of polypropylene in order to facilitate heat adhesion to the plastic film inner layer of the medical fluid bag.

The content of ingredients constituting the outer layer is preferably 20 to 55% by weight of polypropylene-based elastomer and 45 to 80% by weight of polypropylene, based on the total weight of the outer layer. In the case where the content of polypropylene-based elastomer is less than 20% by weight, the flexibility of the outer layer is reduced. In the case where the content of polypropylene-based elastomer is more than 55% by weight, the flexibility is too high to perform continuous operation in a feeding apparatus. Further, in the case where the content of polypropylene is less than 45% by weight, the heat adhesion to the medical fluid bag is deteriorated, and the content of polypropylene-based elastomer is relatively high to deteriorate workability as mentioned above. Further, in the case where the content of polypropylene is more than 80% by weight, the flexibility of outer layer is reduced.

With respect to the medical tube of the present invention, the outer layer preferably consists of 30 to 55% by weight of polypropylene-based elastomer and 45 to 70% by weight of polypropylene; or more preferably comprises 1 to 20% by weight of polyethylene, in addition to 20 to 54% by weight of polypropylene-based elastomer and 45 to 70% by weight of polypropylene, based on the total weight of the outer layer. The composition ratio has the same meaning as mentioned above. The composition ratio is provided only for the purpose of illustrating more preferred range of composition ratio.

Upon adhesion to the medical fluid bag made of polyolefin material, if polyethylene is added in a predetermined amount as described above, the tube may be selectively applied to containers made of more various materials, as compared to the case of using polypropylene only.

In the case where the content of polyethylene is less than 1% by weight, the effect due to its addition is not sufficient. In the case where the content of polyethylene is more than 20% by weight, the effect is no longer increased, whereas heat resistance required for sterilization is deteriorated, and the problems related to relative decrease of the polypropylene content are generated.

It is preferable that the medical tube of the present invention comprises 5 to 10% by weight of the inner layer, 75 to 80% by weight of the intermediate layer, and 15 to 20% by weight of the outer layer, based on the total weight of the tube.

With respect to the inner layer, intermediate layer, and outer layer constituting the medical tube of the present invention, each layer may be formed by blending of the polymers constituting the corresponding layer.

In general, the medical tip is inserted into the medical tube, and binds thereto by heat adhesion without any adhesive. The inner layer of the medical tube of the present invention has excellent properties in the insertion of the medical tip and in the prevention of the tip separation. In addition, the inner layer of the medical tube of the present invention has excellent chemical stability as the parts directly contacted with medical fluid.

Further, the medical tube is inserted into the medical fluid bag and generally fixed by heat adhesion. Thus, the outer layer of the medical tube of the present invention has a property of excel lent heat adhesiveness to the medical fluid bag made of a plastic film.

The polyolefin-based medical tube including the medical tube of the present invention has low yield strength, thereby being prone to necking. Necking is a phenomenon that the diameter of a tube is locally reduced, when the tube is expanded under a suitable stress according to the longitudinal axis of the tube. There is a linear proportional relationship between yield strength and modulus. Therefore, in the present invention, yield strength is increased by increasing modulus of the material, thereby solving the problem.

The medical tube of the present invention is used as a port of the medical fluid bag, a connection line of an infusion set or the like. The infusion set refers to a part or system which is used to convey fluid to a patient's body upon administration of an IV (intravenous) solution in hospital, such as a needle, a tube, a drop chamber, and a spike plastic needle for piercing the fluid bag. The medical tube of the present invention is used to convey a variety of solutions for medical use, in particular, blood.

The medical tube of the present invention may be subjected to a dry orientation process or a wet orientation process in a cooling step after molding. Further, shrinkage problem, which occurs due to memory effect during shipping, storage and use, can be solved by packing the tube with nitrogen after molding and aging.

In the medical tube of the present invention, two or more materials (resin) used for molding of the tube may be blended by melt blending or tumble blending. Further, with respect to the extruder used for molding of the tube, conditions including an outer diameter of die pin and an inner diameter of die bushing are set, considering shrinkage of the tube. The medical tube of the present invention is prepared by extruding the structure of three layers at the same time.

Further, the present invention provides a medical fluid bag set including the medical tube.

### MODE FOR INVENTION

### Examples 1 to 2 and Comparative Examples 1 to 4

### (1) Manufacture of Medical Tube

Materials were blended by melt blending or tumble blending according to the composition and composition ratio described in the following Table 1. With respect to the extruder used for molding of the tube, conditions including an outer diameter of die pin and an inner diameter of die bushing were set, considering shrinkage of the tube, and three layers of inner layer, intermediate layer, and outer layer were extruded at the same time to manufacture the medical tube of Example 1.

The medical tubes of Example 2 and Comparative Examples 1 to 3 were manufactured in the same manner as Example 1, according to the composition and composition ratio described in the following Table 1.

As a tube of Comparative Example 4, a NON-PVC tube by electronic cross-linking of EVA was used.

### (2) Performance Evaluation of Medical Tube

The performance evaluation on the medical tubes manufactured in Examples 1 and 2, and Comparative Examples 1 to 4 was performed as follows, and the results are shown in Tables 1 and 2.

A. Evaluation of Film Compatibility

1) Evaluation Conditions

The manufactured tubes were connected to the port of a medical fluid bag, and then the following evaluation was performed.

The medical tubes were sterilized, and then stored for 5 days or more, followed by the evaluation.

2) Evaluation Items and Method
LEAK: LEAK was observed in the tube and the port.
BENDING: bending of the tube was observed.
DROP separation: the tubes were dropped from a height of 1.5 m in the direction of the port, and then the tip separation was observed.
Separation by force: a hand force was applied to the tips which were not separated in the above test, and then their separation was observed.
Heat resistance: the medical tubes were sterilized at 121° C., and occurrence of bending was observed.
Transparency: after performing the sterilization, transparency was observed with the naked eye.
Flexibility: MODULUS measurement (film type)
Gas-barrier property: OTR measurement (film type)

TABLE 1

| | | | Polypropylene- | | | | | | | | | Separation |
|---|---|---|---|---|---|---|---|---|---|---|---|---|
| | | | based elastomer | | | | | | | | DROP | by |
| No. | Constitution | Ratio | 1 | 2 | 3 | total | PP | PE | LEAK | BENDING | separation | force |
| Exam 1 | inner | 10 | 0 | 5 | 65 | 70 | 30 | 0 | 0 | 0 | 0 | 0 |
| | intermediate | 75 | 0 | 45 | 0 | 45 | 55 | 0 | | | | |
| | outer | 15 | 40 | 0 | 0 | 40 | 50 | 10 | | | | |
| Exam 2 | inner | 10 | 0 | 0 | 65 | 65 | 25 | 10 | 0 | 0 | 0 | 0 |
| | intermediate | 75 | 0 | 50 | 0 | 50 | 50 | 0 | | | | |
| | outer | 15 | 40 | 0 | 0 | 40 | 50 | 10 | | | | |
| Comparative | inner | 10 | 0 | 0 | 30 | 30 | 20 | 50 | 0 | 2.5 | 0 | 1.3 |
| Exam 1 | intermediate | 75 | 0 | 50 | 0 | 50 | 50 | 0 | | | | |
| | outer | 15 | 40 | 0 | 0 | 40 | 50 | 10 | | | | |
| Comparative | inner | 10 | 0 | 0 | 65 | 65 | 25 | 10 | 0 | 4.3 | 0 | 2.9 |
| Exam 2 | intermediate | 75 | 0 | 40 | 0 | 40 | 60 | 0 | | | | |
| | outer | 15 | 40 | 0 | 0 | 40 | 50 | 10 | | | | |
| Comparative | inner | 10 | 0 | 0 | 65 | 65 | 25 | 10 | 0 | 2 | 38 | 32.3 |
| Exam 3 | intermediate | 75 | 0 | 50 | 0 | 50 | 50 | 0 | | | | |
| | outer | 15 | 20 | 0 | 0 | 20 | 50 | 30 | | | | |

| | | | | | | |
|---|---|---|---|---|---|---|
| Comparative | inner | EVA | 0 | 3.1 | 4.9 | 18.2 |
| Exam 4 | outer | PP TERPOLYMER | | | | |

| | |
|---|---|
| Unit: % by weight | Unit: Defective rate % |

In Table 1, PP represents a PP Terpolymer (DAE HAN OIL CHEMICAL Co., Ltd.), PE represents ENGAGE (DOW CHEMICAL), and the polypropylene-based elastomers 1, 2, and 3 were selected from DYNARON (JSR, Japan), HYBRAR (KURARAY, Japan), and KRATON (KRATON).

TABLE 2

| | | | Comparative | |
|---|---|---|---|---|
| Test item | test method | Example 1 | Example 4 | PP MONO |
| Heat | BENDING | Excellent | Defective | Good |
| resistance | | | | |
| (sterilization | | | | |
| at 121° C.) | | | | |
| Transparency | — | Excellent | Defective | Excellent |
| Flexibility | — | Good | Good | Defective |
| Heat | LEAK | 0% | 0% | 9% |
| adhesiveness | | | | |

### INDUSTRIAL APPLICABILITY

The medical tube of the present invention is excellent in transparency, flexibility, heat resistance, and chemical stability, and also has excellent heat adhesiveness to NON-PVC medical fluid bags and gas-barrier property, thereby being very useful in medical fields.

1. A medical tube, comprising
an inner layer comprising 50 to 90% by weight of polypropylene-based elastomer and 10 to 50% by weight of polypropylene, based on a total weight of the inner layer;
an intermediate layer comprising 45 to 55% by weight of polypropylene-based elastomer and 45 to 55% by weight of polypropylene, based on a total weight of the intermediate layer; and
an outer layer comprising 20 to 55% by weight of polypropylene-based elastomer and 45 to 80% by weight of polypropylene, based on a total weight of the outer layer.

2. The medical tube according to claim 1, wherein the inner layer further comprises 1 to 40% by weight of polyethylene, in addition to 50 to 70% by weight of polypropylene-based elastomer and 10 to 30% by weight of polypropylene.

3. The medical tube according to claim 1, wherein the outer layer further comprises 1 to 20% by weight of polyethylene, in addition to 20 to 54% by weight of polypropylene-based elastomer and 45 to 70% by weight of polypropylene.

4. The medical tube according to claim 1, wherein the inner layer consists of 50 to 80% by weight of polypropylene-based elastomer and 20 to 50% by weight of polypropylene, based on the total weight of the inner layer.

5. The medical tube according to claim 1, wherein the outer layer consists of 30 to 50% by weight of polypropylene-based elastomer and 50 to 70% by weight of polypropylene, based on the total weight of the outer layer.

6. The medical tube according to claim 1, wherein the medical tube comprises 5 to 10% by weight of the inner layer, 75 to 80% by weight of the intermediate layer, and 15 to 20% by weight of the outer layer, based on the total weight of the medical tube.

7. The medical tube according to claim 1, wherein the inner layer, the intermediate layer, and the outer layer are formed by blending of the polypropylene-based elastomer and the polypropylene constituting a corresponding layer.

8. The medical tube according to claim 1, wherein the medical tube is used as a port of a medical fluid bag or a connection line of an infusion set, or is used to convey blood.

9. A medical fluid bag set comprising the medical tube of claim 1.

10. The medical tube according to claim 2, wherein the outer layer further comprises 1 to 20% by weight of polyethylene, in addition to 20 to 54% by weight of polypropylene-based elastomer and 45 to 70% by weight of polypropylene.
